(19) United States
(12) Patent Application Publication
(10) Pub. No.: US 2026/0047846 A1
(43) Pub. Date: Feb. 19, 2026

(54) Medical Implant

(71) Applicant: Acandis GmbH, Pforzheim (DE)

(72) Inventors: Andreas Schüßler, Pfinztal (DE); Andrés Braschkat, Bretten (DE)

(21) Appl. No.: 18/804,680

(22) Filed: Aug. 14, 2024

(30) Foreign Application Priority Data
Jun. 23, 2023 (DE) 102023116579.8

(51) Int. Cl. A61B 17/12; A61B 17/00

(52) U.S. Cl. CPC A61B 17/12118; A61F 2/90; A61B 2017/00867; A61B 17/12177

(57) ABSTRACT

A medical implant for the treatment of a bifurcation aneurysm, with a support body that is compressible and expandable, wherein the support body includes a proximal section and a distal section which are connected to each other, wherein the distal section is tubular for anchoring in the vessel, and the proximal section is conical and has an opening angle in relation to the longitudinal axis of the support body, so the proximal section can be positioned in the vessel such that the lesion can be at least partially covered, and the proximal section has a first and a second porosity, wherein the first porosity is greater than the second porosity so that in the implanted state, the vessel and/or the lesion is accessible through the first porosity with a microcatheter, and the blood flow through the second porosity can be at least partially diverted from the lesion.

### CROSS REFERENCE TO RELATED PATENT APPLICATIONS

The present application claims the benefit of and priority to German Patent Application No. 10 2023 116 579.8, filed Jun. 23, 2023, the entire contents of which are incorporated herein for all purposes by reference.

### FIELD

The invention relates to a medical implant for the treatment of a local lesion in a bifurcation of a vessel, more particularly for the treatment of a bifurcation aneurysm, with a support body that is compressible and expandable, wherein the support body comprises a proximal section and a distal section which are connected to each other.

### BACKGROUND

A medical implant according to the preamble of claim 1 is known from DE 102015122679 A1, for example.

For the treatment of vascular lesions, such as aneurysms, for example, medical implants such as flow diverters are used, which reduce the blood flow into the aneurysm, so that the blood present in the aneurysm can coagulate, if necessary supported by an embolisate. The interventional accessibility of the vessels can be restricted by the position of the flow diverter.

### SUMMARY

The invention is therefore based on the task of disclosing a medical implant for the treatment of a lesion in a bifurcation of a vessel, which on the one hand allows the treatment of the lesion and on the other hand enables access to the vessel, more particular branching off arms of the vessel.

According to the invention, this task is solved by a medical implant with the features shown and described herein.

More specifically, this task is solved by a medical implant for the treatment of a local lesion in a bifurcation of a vessel, more particularly for the treatment of a bifurcation aneurysm, with a support body that is compressible and expandable, wherein the support body comprises a proximal section and a distal section which are connected to each other. The distal section is tubular, more particularly cylindrical for anchoring in the vessel. The proximal section is conical and has an opening angle in relation to the longitudinal axis of the support body, so that the proximal section can be positioned in the vessel in such a way that the lesion can be at least partially covered. The proximal section has a first and a second porosity, wherein the first porosity is greater than the second porosity so that in the implanted state, the vessel and/or the lesion is accessible through the first porosity with a delivery device, more particularly a microcatheter, and through the second porosity the blood flow can be at least partially diverted from the lesion.

The invention has various advantages. As the proximal section of the support body is designed conically and has an opening angle in relation to a longitudinal axis of the support body, the proximal section can be positioned in the vessel in such a way that a lesion in a bifurcation of a vessel can at least partially be covered. The conical shape ensures that the proximal section of the support body can contact the vascular wall in the area of the lesion. The area of the conical shape close to the lesion follows the vascular wall in the area of the lesion, so that good wall apposition of the support body and thus good coverage of the lesion is achieved. Here, it is advantageous, but not absolutely necessary, for the lesion to be completely covered.

Furthermore, the conical shape provides a certain anchoring of the proximal section in the area of the bifurcation, as the area of the conical shape away from the lesion can come into contact with the vascular wall in the area of the supplying vessel.

Through the invention, in the bifurcation the lesion can be shielded from the blood flow, wherein the accessibility of the vessels and/or the lesion is largely retained. For this, the proximal section has a first and a second porosity, wherein the first porosity is greater than the second porosity. The second porosity is designed such, that the blood flow can be diverted from the lesion so that the blood present in the lesion can coagulate. The first, greater porosity provides accessibility of the vessel and/or lesion in the implanted state so that a delivery device, more particularly a microcatheter, can be passed through the proximal section. The first porosity is adapted to the delivery device accordingly.

The first and the second porosity of the proximal section fulfil a double function. The conical first section can both deflect the blood flow from the lesion as well as be passed through by the delivery device in order to allow interventional reachability of distal vascular areas.

In the context of the invention, the different porosities of the proximal section should be understood to the effect that on the proximal section, along the longitudinal axis of the support body, at least two areas with a different porosity can be measured. These can transition into each other discretely or continuously. The arrangement and size of the first and the second porosity are adjustable, so that both functions, i.e. the accessibility of the lesion and/or vessels as well as the deflection of the blood flow from the lesion can be fulfilled.

In a particularly preferred form of embodiment, the wall of the proximal section is concave in design so that the wall can be adapted to the curvature of the vessels in a bifurcation. In other words, the wall of the proximal section can be designed concavely in such a way that the curvature of the wall corresponds to the curvature of the vessels. Expressed differently, the wall can preferably be designed in such a way that the wall follows the wall of the curvature. In general it applies that in a vascular bifurcation an angle is formed between branching-off vessels and consequently a curvature at the transition of one branching off vessel into the other. Through a concave wall of the proximal section, optimal adaptation to the vascular anatomy in the aera of a bifurcation or lesion is achievable.

Also preferably, the wall of the proximal section has an angle of curvature of between 70° and 190°, more particularly at least 90°, more particularly at least 110°, more particularly at least 130°, more particularly at least 150°, more particularly at least 170°. Furthermore, the angle of curvature can be, in particular maximally 190°, in particular maximally 180°, in particular maximally 170°. This is to be understood to the effect that the wall is designed in such a way that the wall can be adapted to a curvature that forms in a vessel bifurcation, i.e. between two branching-off vessels that are at an angle of between 70° and 190° to each other. Such a curvature angle range is particularly advantageous, for example in bifurcations of the middle cerebral artery, the basilar artery, the internal carotid artery or the anterior cerebral artery.

In a further preferred form of embodiment, the first porosity is arranged at the proximal end, and the second porosity at the distal end of the proximal section. Through the position of the first porosity, which is greater than the second porosity, at the end of the proximal section, the blood flow in the vessels can be maintained. In other words, the blood can flow through the first porosity, so that adequate supply of following vessels and/or tissue areas can be assured. Moreover, through the position of the second or lower porosity at the distal end of the proximal section, at least partial coverage of the lesion can be achieved. Through this, the blood flow can be diverted from the lesion. Consequently, the blood flow in the lesion is reduced, through which the blood present in the lesion can coagulate.

Also preferably, the porosity changes continuously in the axial direction of the proximal section. In other words, the transition from the first to the second porosity can be continuous. Furthermore, the porosity within the first and second porosity can change continuously. Particularly preferably, the porosity of the proximal section is designed to decrease in the distal direction. This means that the greater porosity is preferably present at the proximal end, whereas the distal end has lowest porosity of the proximal section. In this way, coverage of the lesion can be achieved without restricting the blood flow into the branching-off vessels. Furthermore, it is advantageous that the blood flow in the supplying vessel of the bifurcation is is not, or is hardly, impaired.

Furthermore, the distal section advantageously has a different porosity from the proximal section. Particularly preferably the distal section has a lower porosity than the proximal section. Here, it is of advantage that the distal section ensures good anchoring in the vessel, more particularly in a vessel branching off from a bifurcation. For this, the distal section is preferably designed in such a way that the support body is in contact with the vascular wall and therefore good wall adaptation is present.

Preferably the proximal section has a larger cross-sectional diameter than the distal section. Through this it is possible to achieve optimal coverage of a lesion in a bifurcation of a vessel. The larger cross-sectional diameter of the proximal section allows good adaptation to the anatomy in the area of a bifurcation. In this way, secure anchoring of the support body close to the lesion can be achieved.

In a further preferred form of embodiment, the proximal section comprises a proximal longitudinal end and the distal section a distal longitudinal end of the support body, wherein the cross-sectional diameter of the proximal longitudinal end is at least 1.5 times, preferably 2 times, more particularly 2.5 times, in particular 3 times greater than the cross-sectional diameter of the distal longitudinal end. Here, it is of advantage that the size or the cross-sectional diameter of the proximal end is selectable so that the implant is adaptable to different anatomies in the area of the bifurcation.

Also preferably, the proximal section has an opening angle of at least 45°, more particularly 50°, more particularly 55°, more particularly 60°, more particularly 65, more particularly 70°, more particularly 75°. The opening angle is formed between the longitudinal axis and the wall of the proximal section. In other words, the conical shape of the proximal section can be described by the opening angle. Particularly preferably, the opening angle is 45°, 50°, 55°, 60°, 65°, 70°, 75° or 80°. Alternatively it is possible for the proximal section to have a different opening angle. The conicity of the proximal section is therefore advantageously adaptable to different anatomies.

In addition, in the axial direction the proximal section has a length of at least 4 mm, more particularly 5 mm, more particularly 6 mm, more particularly 7 mm, more particularly 8 mm, more particularly 9 mm, more particularly 10 mm, more particularly 12 mm. Here, the length of the proximal section describes the distance between the proximal and distal end of the proximal section which is formed along the longitudinal axis of the support body. Particularly preferably, the length of the proximal section is 4 mm, 5 mm, 6 mm, 7 mm, 8 mm, 9 mm, 10 mm, 11 mm or 12 mm. Alternatively, it is possible that the proximal section has a different length. Through this, the length of the proximal section can be adapted to different anatomies in the area of a bifurcation.

Shown as examples in the following table are preferred parameter ranges of the length, cross-sectional diameter and opening angle of the proximal section:

| Length of | Cross-sectional | Opening angle α |
|---|---|---|
| the proximal | diameter of the | of the proximal |
| section | proximal longitudinal end | longitudinal end |
| 4 mm to 6 mm | 5 mm to 10 mm | 50° to 60° |
| 5 mm to 8 mm | 6 mm to 13 mm | 55° to 65° |
| 8 mm to 10 mm | 7 mm to 15 mm | 60° to 70° |
| 10 mm to 12 mm | 8 mm to 18 mm | 65° to 80° |

In general, statements regarding to the geometry of the implant, such as the cross-sectional diameter and/or the opening angle, relate to the implants in the expanded rest state, i.e. in the state with no forces exerted on it.

The support body advantageously has a braided structure made of wires which are each wound around a longitudinal axis of the braided structure and cross over and under each other, wherein the braided structure has between 24 and 64, more particularly 48 wires. In other words, the support body can be produced by the braiding of wires. The support body can thus be formed of wires or consist of wires. The number of wires can be adapted to the individual application. For example, the radial force and thus the supporting effect of the implant can be influenced by the number of wires.

It is also possible to produce the implant by the braiding of wires on a braiding mandrel. The braiding mandrel can be individually tailored to the patient. By way of various imaging diagnostic procedures, such as MRI or CT procedures, the vessel or the lesion to be treated can be visualised. On the basis of these images, it is possible to produce a braiding mandrel that is adapted to a patient's anatomy, The patient-customised braiding mandrel is then used as a core on which the wires are wound in a helical manner.

Particularly preferably, the wires have a braid angle in relation to the longitudinal axis of the support body, wherein the wires in the proximal section have a different braid angle from the wires in the distal section. In other words, the braid angle is determined by the inclination of the wires relative to the longitudinal axis L. Here it is advantageous that the porosity of the support body can be adjusted by the braid angle in a simple way. Particularly preferably, the wires in the proximal section have a smaller braid angle than the wires in the distal section. Consequently, the porosity in the distal section is lower than in the proximal section. Through this, a good support effect can be achieved in the distal section.

If, as in a preferred form of embodiment of the invention, the support body is made of wires, the porosity describes the ratio of the projected wire surface (minus the cross-over area) to the entire surface area of the support. More particularly, the porosity in the proximal and/or distal section of the support body can be influenced by the number of wires, the wire diameter, the outer diameter of the support body and/or the braid angle.

Preferably the wires in the distal section have a braid angle of between 85° and 60°, more particularly 75°. A braid angle of approximately 75° in the distal section is particularly preferred, as the porosity can be adjusted in such a way that a good support effect and thereby good anchoring in the vessel can be guaranteed.

In a further preferred form of embodiment, the wires at the proximal longitudinal ends of the braided structure form closed loops. Here it is of advantage that the loops provide an increased radial force on the proximal end of the braided structure, through which secure anchoring of the support body close to the lesion is guaranteed. Also preferably, the closed loops can form an increase in the diameter of the proximal longitudinal end of the support body. Through such radial expansion of the proximal longitudinal end, the risk of migration of the stent, for example, can be reduced.

It is also preferable that the wires are made of a core material visible in X-rays, more particularly platinum or an platinum alloy, and shape memory material, in particular a nickel-titanium alloy, wherein the core material makes up a surface area of between 10% and 50%, more particularly between 20% and 30%, of the cross-sectional area of the wire. The proportion of the core material in the cross-sectional area of the wire is, for example, dependent on the wire diameter or can adapted to the wire diameter.

It is advantageous that through the core material the wires are optimally visible in X-rays. In this way, for example, an operator can easily determine the position of the implant during the placement of the implant or in the implanted state. Particularly preferably, the core material is made of platinum or a platinum alloy, as these materials have optimum radiopacity and are consequently visible in X-rays. As a result, it is not necessary to use additional marker elements, such a marker sleeve, that can be applied or crimped into the wire. However, the use of additional marker elements is not ruled out. If is, for example, possible to fasten a marker element onto the proximal section in such a way that in the implanted state it is positioned in the bifurcation or at the transition between branching off vessels. Through this, correct placement of the implant, particularly of the proximal section, can assured through optimum X-ray visibility.

In addition the X-ray visible core material can be surrounded by a shape memory material. In other words, the surface of the wires is made of a shape memory material. Here, it is advantageous that through the shape memory material, the implant is self-expanding or has self-expanding properties.

Such wires that are made of an X-ray visible core material that is surrounded by a shape memory material, are generally known as DFT wires.

The wires preferably have a wire diameter of between 20 μm and 50 μm, more particularly 30 μm. These wire diameters advantageously influence the radial force of the support body, through which an optimal expansion and compression behaviour can be achieved. In addition, the porosity of the support body can be influenced by the wire diameter.

Also advantageously, the support body has an antithrombogenic coating, which, in particular, comprises heparin and/or fibrin. In other words, the support body can be coated with a material that has antithrombogenic properties. The coating can develop an anticoagulant effect so that the adhesion of blood proteins is favourably influenced. In this way, a layer of cells, in particular endothelial cells, can form over the support body. The material of the support body is thus masked by a blood protein layer, which also prevents or reduces the depositing of thrombocytes and coagulation proteins, for example fibrinogen, which are mainly responsible for the formation of thrombi. Overall, a reduction in the tendency to form thrombi is thus brought about. Particularly preferably the antithrombogenic coating comprises heparin and/or fibrin, wherein heparin can be covalently bonded to fibrin.

### BRIEF DESCRIPTION OF THE DRAWINGS

This invention will be explained further by way of an example of embodiment in connection with the drawing with further details.

In this

FIG. 1 shows an example of embodiment according to the invention of a medical implant for the treatment of a local lesion; and

FIG. 2 show the medical implant according to FIG. 1 in the implanted state in the area of a bifurcation of a vessel.

### DESCRIPTION OF AN EMBODIMENT

In the following, the same reference numbers are used for parts that are the same or function in the same way.

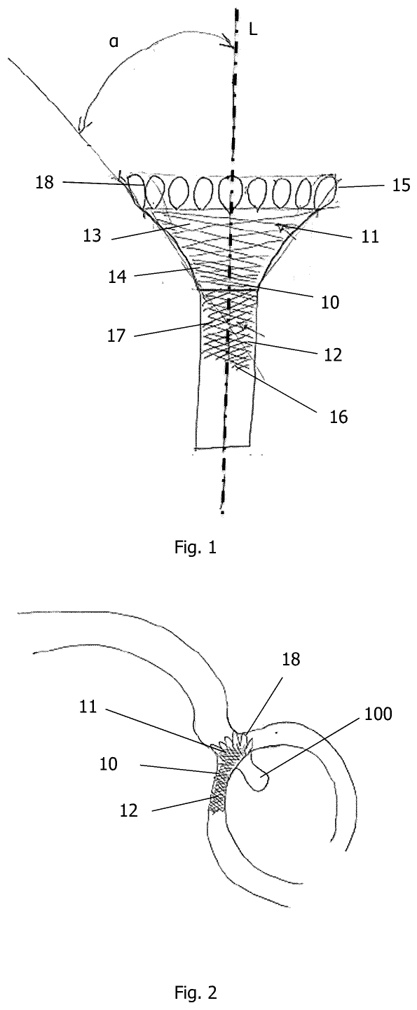

FIG. 1 shows an example of embodiment according to the invention of a medical implant for the treatment of a local lesion 100 in a bifurcation of a vessel. This involves the use of the implant for the treatment of a bifurcation aneurysm. Other uses are also conceivable. In general, the implant is therefore suitable for the treatment of vascular lesions 100 located in the area of a vascular bifurcation.

The example of embodiment depicted in FIG. 1, shows the implant in the expanded resting state, i.e. in the state of the support body 10 without forces exerted on it. This means that no external force are acting on the implant and this is not in use.

In FIG. 2, the implant is shown in the implanted state. It can be seen that the implant is positioned in a bifurcation of a vessel in order to cover a vascular lesion 100. In the specific example according to FIG. 2, the lesion 100 or the neck of the lesion 100 is completely covered by the support body 10. Alternatively it is possible that the implant only partially covers the lesion 100.

FIG. 2 also shows that one single implant is positioned in a bifurcation of a vessel. Alternatively, it is possible to position two implants according to the invention in a bifurcation, for example.

In FIG. 1 it can be seen that the implant has a support body 10 with a proximal and a distal section 11, 12 which are connected to each other. In other words, the proximal section 11 transitions into the distal section 12. In the transitional area, the proximal and distal section 11, 12 have the same cross-sectional diameter.

From FIG. 1 it can also be seen that the distal section 12 is designed cylindrical. In other words, the distal section 12 has an constant cross-sectional diameter. A different geometry of the distal section 12 is conceivable. Thus, the distal section 12 can, for example, have a concave wall in order to improve adaptation to the vascular anatomy.

FIG. 1 also shows that the proximal section 11 is conical. In other words, the proximal section 11 is conically widened. The proximal section 11 has a geometry that tapers in the distal direction. It can also be seen that the proximal section 11 has an opening angle α in relation to the longitudinal axis L. This means that the conical shape of the proximal section 11 can be described or is defined by the opening angle α. Through the conical shape, the proximal section 11 is, as shown in FIG. 2, positioned in a vascular bifurcation in such a way that a lesion 100 is covered, in order to reduce the blood flow into the lesion 100. The lesion 100 can be completely or partially covered by the implant.

From FIG. 1 it is also evident the proximal section 11 has a first and a second porosity 13, 14, wherein the first porosity 13 is greater than the second porosity 14. Here it can be seen that on the proximal section 11, along the longitudinal axis L there are areas that have a different porosity. This is to be understood to the effect that on the proximal section 11 at least two porosities can be measured, which differ from each other.

FIG. 2 also shows that the vessels and/or the lesion 100 are accessible through the first porosity 13 with a delivery device. Thus, the first porosity 13 allows, for example, the interventional reachability of the vessels of the bifurcation and the lesion 100 itself with the help of a microcatheter. Furthermore, it can be seen in FIG. 2 that though the second porosity 14 the blood flow is diverted from the lesion 100 in such a way that the blood flow into the lesion 100 is reduced as a result of which the blood present in the lesion 100 can coagulate. Consequently, the first and the second porosity are adjusted or selected so that both functions, i.e. the accessibility of the lesion and/or vessels as well as the deflection of the blood flow from the lesion can be fulfilled.

FIG. 1 also shows that the wall of the proximal section is concave in order to adapt the wall to the curvature of the vessels in a bifurcation. It can be seen that the wall has a bulge or is bulged. FIG. 2 also illustrates that a concave wall of the proximal section enables optimal adaptation to the anatomy.

In the example of embodiment according to FIG. 1, the wall of the proximal section has an angle of curvature of between 90° and 170°. Other angle ranges are also conceivable. Concretely, the curvature of wall can be adapted to the specific case or to the anatomy of a bifurcation.

In FIG. 1 it is shown that the implant or the proximal and distal section 11, 12 of the support body 10 are designed to be rotationally symmetrical. It is conceivable that the implant has a different geometry. The implant or the proximal and/or distal section can therefore have a curved geometry. This is to be understood to the effect that the longitudinal axis L of the implant has a curvature, whereby the proximal and distal section 11, 12 are aligned along this curvature.

FIG. 1 shows that the first porosity 13 is arranged at the proximal end and the second porosity 14 at the distal end of the proximal section 11. Consequently, the proximal section 11 has a lower porosity in its distal area than in its proximal area. It can also be seen that the first porosity 13 transitions into the second porosity 14 or vice-versa.

In the example of embodiment according to FIG. 1, the porosity changes continuously in the axial direction of the proximal section 11. It can be seen that the proximal section 11 has the greatest porosity at its proximal end, wherein the porosity decreases continuously from proximal to distal. It is also possible for the proximal section 11 to comprise local areas with a great or low porosity which are at a distance from one another or do not continuously transition into each other.

From FIG. 1 it can also be seen that the distal section 12 has a lower porosity than the proximal section 11. It can also be seen that the distal section 12 of the support body 10 has denser structure than the proximal section 11. It is also conceivable that the distal and proximal section 11, 12 have the same porosity. This means that the porosity of the distal section 12 can correspond with the first or second porosity 13, 14 of the proximal section 11. Moreover, the porosity of the distal section 12 can be greater than the porosity of the proximal section 11.

From FIG. 1 it can be seen that the proximal section 11 has a greater cross-sectional diameter than the distal section 12. In other words, the proximal section 11 has a greater cross-sectional area than the distal section 12. Here, due to its conical shape, the proximal section 11 has a greater cross-sectional diameter at every position along the longitudinal axis L than the distal section 12. It can be seen that the cross-sectional diameter increases in the proximal direction of the proximal section 11.

FIG. 1 shows that the proximal section 11 comprises a proximal longitudinal end 15 and the distal section 12 a distal longitudinal end 16 of the support body 10. In the shown example of embodiment, the cross-sectional diameter of the proximal longitudinal end 15 is at least 1.5 times greater than the cross-sectional diameter of the distal longitudinal end 16. Other size ratios between the proximal and distal longitudinal end 15, 16 are conceivable. For example, the proximal longitudinal end 15 can be at least 2 times, preferably 2.5 times or 3 times greater than the cross-sectional diameter of the distal longitudinal end 16.

In FIG. 1 it is also shown that the proximal section 11 has an opening angle α of at least 45°. It can be seen that the opening angle α is formed between the longitudinal axis L and the wall of the proximal section 11. It is also conceivable that the proximal section 11 has a different, more particularly larger, opening angle α. For example, the opening angle α can be 50°, 55°, 60°, 65°, 70°, 75° or 80°.

In addition, in the example of embodiment according to FIG. 1, in the axial direction the proximal section 11 has a length of at least 4 mm. The length of the proximal section 11 is taken to mean the length between the proximal and distal longitudinal end 15, 16 of the proximal section 11, which is measured along the longitudinal axis L. For example, the length of the proximal section 11 is 5 mm, 6 mm, 7 mm, 8 mm, 9 mm, 10 mm, 11 mm or 12 mm. Other lengths of the proximal section 11 are conceivable.

It can also be seen from FIG. 1 that the support body 10 has a braided structure made of wires 17 which are each wound around a longitudinal axis L of the braided structure and cross over and under each other. In the specific example of embodiment, the braided structure is formed of 48 wires 17. A different number of wires 17 is conceivable. Thus, the braided structure can comprise between 24 and 64 wires 17. The braided structure is produced by braiding the wires 17, wherein the wires 17 form meshes of the braided structure.

The braided structure can be produced according to different braiding types. For producing the braid pattern, a method can be used that is known as plying. Plying means that at least two filaments are placed together in the same plane. The implant can thus comprise a wall, braided at least in parts, which has strands formed of several filaments, which are braided with each other and/or with individual filaments, wherein at least one strand comprises several filaments which are all arranged next to each other in the plane extending along the wall, and the filaments of a strand have different diameters or the diameters of the filaments vary between several strands and are the same within a strand. Plying, i.e. the formation of flat strands with different numbers of filaments, makes it possible, for example, to achieve high radial forces at the ends of the implant.

The wire(s) can be braided in a 1 over 1 or 1 over 2 arrangement. Other arrangements are possible.

In addition, the implant can be manufactured through the twisting of wires. In braiding, the term twisting can be taken to mean the mutual winding of two or more wires that mutually wrap around each other at an angle of 360°.

In FIG. 1 it can also be seen that the wires 17 have a braid angle in relation to the longitudinal axis L of the support body 10. It can be seen that the braid angle is enclosed by the longitudinal axis L and a wire section of the wire 17 that intersects the longitudinal axis L. The wires 17 in the proximal section 11 have a smaller braid angle than the wires 17 in the distal section 12. It is also possible that the wires 17 in the proximal and distal section 11, 12 have the same braid angle. Alternatively, the wires 17 in the proximal section 11 have a greater braid angle than in the distal section 12.

In the specific example of embodiment, the wires 17 in the distal section 12 have a braid angle of 75°. Another braid angle is conceivable. In the distal section, the braid angle can be between 60° and 75° for example.

FIG. 1 also shows that the wires 17 at the proximal longitudinal end 15 of the braided structure form closed loops 18. The wires 17 at the proximal longitudinal end 15 are turned around in such a way that end loops 18 are formed. In other words, the wires 17 that form the braided structure, are helically wound about the longitudinal axis L, turned around at the proximal longitudinal end 15 and then wound helically in the opposite direction about the longitudinal axis L. Through this, open wire ends are formed at the distal longitudinal end 16. More specifically, for example, 24 loops 18 are formed at the proximal longitudinal end 15 if 24 wires 17 are used. As the 24 wires 17 for forming the end loops 18 are turned around, at the distal longitudinal end 16 of the braided structure 48 wire ends can thus be seen. The end loops 18 are arranged next to each other in the circumferential direction of the braided structure.

It is also possible that the end loops 19 are in the form of multiple loops or double loops. Here, at least two loops 18 are arranged one behind the other in the longitudinal direction of the braided structure. If a multiple loop is configured as a double loop, the end loop has an inner and an outer loop. In this way a first wire can form a first loop and a second wire a second loop, wherein the first loop and the second loop are arranged at a distance from one another. Through multiple loops, the anchoring of the implant in a bifurcation of a vessel can be further improved.

The wires 17 in the example of embodiment according to FIG. 1 are made of core material visible in X-rays, such as platinum or a platinum alloy and shape memory material, such as a nickel-titanium alloy. Specifically, the core material makes up a surface area of between 20% and 30% of the wire. Another surface area is conceivable. For example, the core material can have a surface area of between 10% and 50% of cross-section area of the wire 17. It is conceivable that the wires are made of another material. Thus, the wires can, for example, be made of a cobalt-chromium alloy or a comparable material.

In addition, the wires 17 have a wire diameter of 30 μm. Other wire diameters are possible. For example, the wire diameter can be between 20 μm and 50 μm.

In the example of embodiment according to FIG. 1, the support body 10 has an antithrombogenic coating. More specifically, the support body 10 is covered with a coating of heparin and fibrin, wherein heparin is covalently bonded to fibrin. Other coatings that give the support body 10 antithrombogenic properties are conceivable. Thus, the coating can comprise fibronectin, for example.

Applicable for all forms and examples of embodiment is that they can be delivered and/or positioned in the vessel by means of a suitable delivery system, for example, a catheter. For this, different catheter systems can be used for different sizes or cross-sectional diameter of the implant.

### LIST OF REFERENCES

10 Support body
11 Proximal section
12 Distal section
13 First porosity
14 Second porosity
15 Proximal longitudinal end
16 Distal longitudinal end
17 Wire
18 Loops
100 Lesion
α Opening angle
L Longitudinal axis

What is claimed is:

1. A medical implant for treatment of a local lesion in a bifurcation of a vessel with a support body that is compressible and expandable, wherein the support body comprises:
a proximal section and a distal section which are connected to each other, wherein the distal section is tubular for anchoring in the vessel, and the proximal section is conical and has an opening angle (a) in relation to the longitudinal axis (L) of the support body, so that the proximal section can be positioned in the vessel in such a way that the lesion can be at least partially covered, and the proximal section has a first porosity and a second porosity, wherein the first porosity is greater than the second porosity so that in an implanted state, the vessel and/or the lesion is accessible through the first porosity with a delivery device and blood flow through the second porosity can be at least partially diverted from the lesion.

2. The medical implant according to claim 1, wherein a wall of the proximal section is concave so that the wall can be adapted to a curvature of the vessel in the bifurcation.

3. The medical implant according to claim 1, wherein a wall of the proximal section has an angle of curvature of between 70° and 190°.

4. The medical implant according to claim 1, wherein the first porosity is arranged at a proximal end and the second porosity at a distal end of the proximal section.

5. The medical implant according to claim 1, wherein a porosity changes continuously in an axial direction of the proximal section.

6. The medical implant according to claim 1, wherein the distal section has a different porosity from the proximal section.

7. The medical implant according to claim 1, wherein the proximal section has a greater cross-sectional diameter than the distal section.

8. The medical implant according to claim 1, wherein the proximal section further comprises a proximal longitudinal end and the distal section a distal longitudinal end of the support body, wherein a cross-sectional diameter of the proximal longitudinal end is at least 1.5 times greater than a cross-sectional diameter of the distal longitudinal end.

9. The medical implant according to claim 1, wherein the opening angle (α) is at least 45°.

10. The medical implant according to claim 1, wherein in an axial direction the proximal section has a length of at least 4 mm.

11. The medical implant according to claim 8, wherein the support body has a braided structure made of wires which are each wound around a longitudinal axis of the braided structure and cross over and under each other, wherein the braided structure has between 24 and 64 wires.

12. The medical implant according any to claim 11, wherein the wires have a braid angle in relation to the longitudinal axis (L) of the support body, wherein the wires in the proximal section have a different braid angle than the wires in the distal section.

13. The medical implant according claim 12, wherein the wires in the distal section have a braid angle of between 85° and 60°.

14. The medical implant according to claim 11, wherein the wires at the proximal longitudinal end of the braided structure form closed loops.

15. The medical implant according to claim 11, wherein the wires are made of a core material visible in X-rays and shape-memory material, wherein the core material makes up a surface area of between 10% and 50% of the cross-sectional area of the wire.

16. The medical implant according to claim 11, wherein the wires have a wire diameter of between 20 μm and 50 μm.

17. The medical implant according to claim 1, wherein the support body has an antithrombogenic coating which comprises heparin and/or fibrin.
